(19) United States
(12) Patent Application Publication
(10) Pub. No.: US 2026/0144657 A1
(43) Pub. Date: May 28, 2026

(54) CATHETERS AND RELATED SYSTEMS AND METHODS OF PERFORMING A MEDICAL INTERVENTION

(71) Applicant: Sonorous NV, Lake Forest, CA (US)

(72) Inventors: Russel Corvese, Mission Viejo, CA (US); Jake Le, Lake Forest, CA (US); Dave Ferrera, Coto De Caza, CA (US)

(21) Appl. No.: 18/956,826

(22) Filed: Nov. 22, 2024

(51) Int. Cl. A61F 2/95; A61M 25/00

(52) U.S. Cl. CPC A61F 2/95; A61M 25/0052; A61M 25/0053

(57) ABSTRACT

An example catheter tube includes an inner conduit and a reinforcement structure. The reinforcement structure includes a coiled wire surrounding the inner conduit and a braided wire assembly surrounding the coiled wire. The reinforcement structure is configured to cause the inner conduit to resist radial deformation as a medical device is moved through the inner conduit. An example medical intervention system includes the example catheter and an example medical device that can be deployed and implanted using the catheter. An example method of performing a medical intervention includes moving the medical device through the inner conduit of the catheter tube, and, while the medical device is moving through the inner conduit, causing the inner conduit to resist radial deformation with the reinforcement structure of the catheter tube.

### TECHNICAL FIELD

This disclosure relates to catheters, such as those used to deliver stents and other medical devices to a target location within a patient's body. This disclosure further relates to associated systems and methods of using such catheters to perform a medical intervention.

### BACKGROUND

Stents may be used to open or widen passageways within the body to treat various medical conditions. In some cases, a catheter may be used to deploy such devices to a desired location within the body for treatment. Cerebral venous sinus stenosis is a disease that obstructs venous blood outflow in the cerebral dural venous sinuses and may be benefit from the implantation of a stent that resolves the obstruction. In some examples, catheters used to deploy such stents may suffer from drawbacks, such as kinking, lack of flexibility in navigating curved regions within the body, and tubular wall damage caused by a stent as the stent is advanced through the catheter.

### SUMMARY

This disclosure relates to catheters, such as those used to deliver stents and other medical devices to a target location within a patient's body. Such catheters include a catheter tube that is designed to resist radial deformation as a medical device is moved through the catheter for optimal performance of the delivery process. This disclosure further relates to associated systems and methods of using such catheters to perform a medical intervention, such as deploying and implanting a braided stent at a portion of a blood vessel within the patient's neurovasculature to treat pulsatile tinnitus.

The catheters discussed herein provide several advantages. For example, a catheter tube of an example catheter includes an inner conduit having an inner surface that is relatively smooth, enabling substantially damage-free movement of the medical device through the lumen of the tube. The length of the tube may be selected to accommodate a patient's anatomical variations and may additionally be selected based on operational preferences of a physician utilizing the catheter.

The catheter tube also includes a dual-layer reinforcement structure that provides the catheter tube with resistance to radial deformation as the medical device is moved axially through the inner conduit and exerts outwardly directed radial forces against an inner surface of the inner conduit. Together, a coiled wire and a braided wire assembly of the reinforcement structure advantageously improve the performance of the methodology for delivering the medical device by minimizing stretching and compression of the inner conduit while the medical device is advanced through the inner conduit of the catheter tube. For example, while the medical device is advanced through the tube, the coiled wire serves to maintain a substantially circular cross-sectional shape of the inner conduit. During manufacture of the catheter tube, the braided wire assembly may be applied directly onto the coiled wire via a braiding machine, which imparts a relatively strong internal tension to the braided wire assembly. Such internal tension aids the coiled wire in resisting radial deformation.

The catheter tube also includes multiple marker bands that are spaced at strategic axial positions along a central axis of the tube and that are visible during medical imaging. Accordingly, the marker bands aid in accurate visual estimation of the distal and proximal ends of the medical device relative to the target location. For example, the marker bands improve a physician's ability to estimate a landing position of the medical device when the device is fully deployed at the target location.

The catheter tube also includes an outer layer that is formed as a hydrophilic coating. The outer layer is fused over the reinforcement structure, the marker bands, and an outer surface of the inner conduit to encapsulate and seal these components to form the tube. Furthermore, the outer layer is formed from one or more relatively soft polymer materials that provide only a minimum resistance to bending. The combined attributes of the inner conduit, reinforcement structure, and outer layer provide the catheter tube with a construction that has a very low stiffness in flexion while maintaining a strong resistance to radial deformation.

In one aspect, a catheter tube includes an inner conduit and a reinforcement structure. The reinforcement structure includes a coiled wire surrounding the inner conduit and a braided wire assembly surrounding the coiled wire. The reinforcement structure is configured to cause the inner conduit to resist radial deformation as a medical device is moved through the inner conduit.

In some embodiments, the catheter tube is a 5 French catheter tube.

In some embodiments, a hardness of the inner conduit varies along a length of the inner conduit.

In some embodiments, the coiled wire has a pitch of about 0.15 mm to about 0.20 mm.

In some embodiments, the braided wire assembly includes multiple wires that overlap each other in regular pattern to form multiple openings.

In some embodiments, the multiple wires includes 16 wires.

In some embodiments, the braided wire assembly has a braid density of 65 pics per inch (PPI) to 75 PPI.

In some embodiments, the catheter tube further includes one or more marker bands that are configured to provide visualization of the catheter tube while the catheter tube is disposed within a patient's body.

In some embodiments, the one or more marker bands include three marker bands that are equally spaced from one another.

In some embodiments, the one or marker bands overlap the reinforcement structure.

In some embodiments, each of the one or more maker bands includes one or more radiopaque materials.

In some embodiments, the catheter tube further includes an outer layer that encapsulates the reinforcement structure and an outer surface of the inner conduit.

In some embodiments, the outer layer includes a coating.

In some embodiments, the inner conduit defines a single lumen that is sized to accommodate the medical device.

In another aspect, a medical intervention system includes a medical device and a catheter tube. The catheter tube includes an inner conduit and a reinforcement structure. The reinforcement structure includes a coiled wire surrounding the inner conduit and a braided wire assembly surrounding the coiled wire. The reinforcement structure is configured to cause the inner conduit to resist radial deformation as the medical device is moved through the inner conduit.

In some embodiments, the medical device includes a stent that is configured to be implanted at a target location within a patient's body.

In some embodiments, the target location includes a portion of a blood vessel within the patient's neurovasculature.

In some embodiments, the medical intervention system further includes a navigation device sized to be positioned within the catheter tube.

In some embodiments, the navigation device includes a main shaft and a radiopaque distal section.

In some embodiments, the radiopaque distal section includes a substantially cylindrical distal shaft and a tapered distal tip that is configured to extend from a distal end of the catheter tube while the catheter tube is moved through a patient's body.

In another aspect, a method of performing a medical intervention includes moving a medical device through an inner conduit of the catheter tube, and, while the medical device is moving through the inner conduit, causing the inner conduit to resist radial deformation with a reinforcement structure of a catheter tube, wherein the reinforcement structure includes a coiled wire surrounding the inner conduit and a braided wire assembly surrounding the coiled wire.

In some embodiments, the method further includes moving the medical device past a distal end of the catheter tube to implant the medical device at a target location within a patient's body.

In some embodiments, the target location includes a portion of a blood vessel of the neurovasculature.

In some embodiments, the medical device includes a stent.

In some embodiments, the catheter tube is a 5 French catheter tube.

In some embodiments, the method further includes causing the inner conduit to maintain a substantially circular cross-sectional shape while the medical device is moved through the inner conduit.

In some embodiments, the method further includes visualizing the catheter tube while the catheter tube is within a patient's body.

In some embodiments, the catheter tube further includes one or more radiopaque marker bands.

In some embodiments, the method further includes, prior to moving the medical device through the inner conduit of the catheter tube: placing a navigation device within the catheter tube and guiding the catheter tube to a target location within the patient's body using the navigation device.

In some embodiments, the navigation device includes a main shaft and a radiopaque distal section, and the radiopaque distal section includes a substantially cylindrical distal shaft and a tapered distal tip that is configured to extend from a distal end of the catheter tube while the catheter tube is in use.

The details of one or more embodiments are set forth in the accompanying drawings and description. Other features, aspects, and advantages of the embodiments will become apparent from the description, drawings, and claims.

### DESCRIPTION OF DRAWINGS

FIG. 1 is a top view of an example catheter system that includes a catheter and a navigation device, where the catheter includes a catheter tube and an access hub.

FIG. 2 is a cross-sectional view of the catheter tube of FIG. 1.

FIG. 3 is a side view of a distal portion of the catheter tube of FIG. 1, including a reinforcement structure with a coiled wire and a braided wire assembly.

FIG. 4 is side view of a portion of a coiled wire of a reinforcement structure of the catheter tube of FIG. 1.

FIG. 5 is side view of a portion of a braided wire assembly of a reinforcement structure of the catheter tube of FIG. 1.

FIG. 6 is a side perspective view of an example medical device that can be delivered to a target location using the catheter of FIG. 1.

FIG. 7 is a front perspective view of the medical device of FIG. 6.

FIG. 8 is a side view of an example insertion device for inserting the medical device of FIG. 6.

FIG. 9 is a side view illustrating the medical device of FIG. 6 and the insertion device of FIG. 8 loaded into the catheter tube of FIG. 1 for implantation.

FIG. 10 is a side view illustrating a connection between the medical device of FIG. 6 and the insertion device of FIG. 8.

FIG. 11 is a flow chart illustrating an example method of implanting a medical device.

FIG. 12 is a diagram that illustrates implantation of the medical device of FIG. 6.

FIG. 13 depicts an example medical image of the medical device of FIG. 6 being implanted within a target blood vessel.

FIG. 14 is a flow chart illustrating an example method of performing a medical intervention.

### DETAILED DESCRIPTION

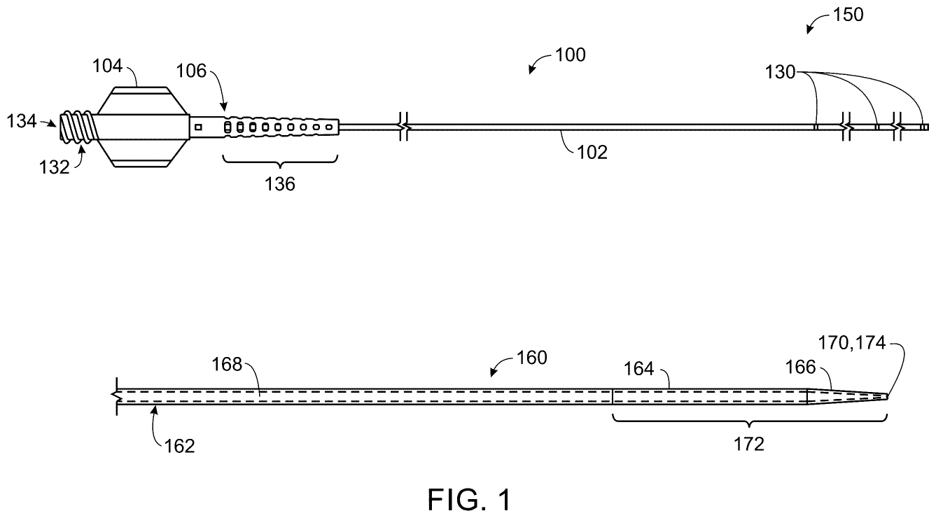

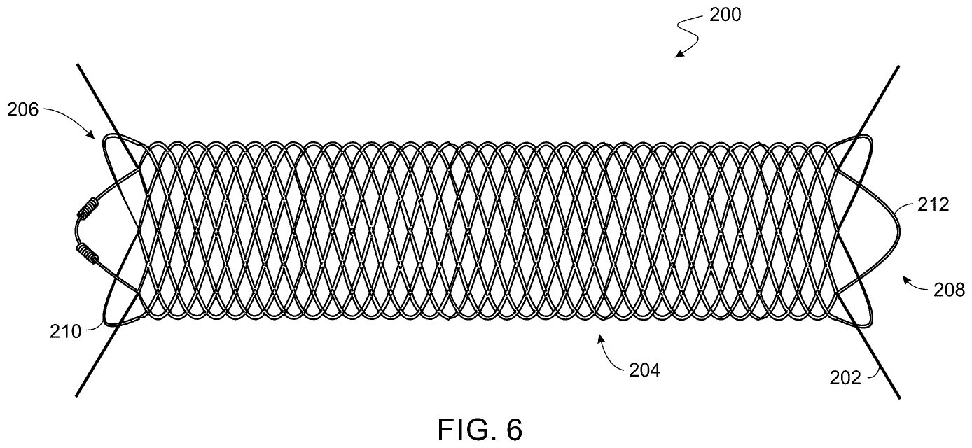

FIG. 1 illustrates an example catheter 100 by which a medical device can be delivered to and deployed at a target location within a patient's body. The catheter 100 is insertable into and navigable through one or more blood vessels of the patient's vascular system to position a distal end of the catheter 100 at the target location, and the medical device is movable axially through the catheter 100 to be deployed outside of the distal end of the catheter 100 and implanted at the target location. In some examples, the target location is a portion of a blood vessel of the patient's neurovasculature (e.g., vasculature located within the head and neck) or a portion of a blood vessel of the patient's peripheral vasculature. In some examples, the medical device is an interventional device, such as a microcatheter, a guide wire, or a braided stent, as will be discussed in more detail below with respect to FIGS. 6-13.

The catheter 100 includes a tube 102 and an access hub 104 that is coupled to a proximal end 106 of the tube 102 at a mating interface. In some embodiments, the catheter 100 is a 5 French catheter. The access hub 104 of the catheter 100 may be a luer fitting or another type of fitting that facilitates attachment or other coupling of accessory devices to the tube 102 at a connection feature 132 (e.g., a threaded profile or another type of connection mechanism). The access hub 104 includes an entry port 134 by which fluids can be infused into the tube 102 or the medical device can be inserted into the tube 102. Additionally, the access hub 102 includes a tapered, slotted portion 136 that extends over the proximal end 106 of the tube 102. Slots along the portion 136 provide strain relief so as to prevent kinking of the tube 102 near the proximal end 106 of the tube 102.

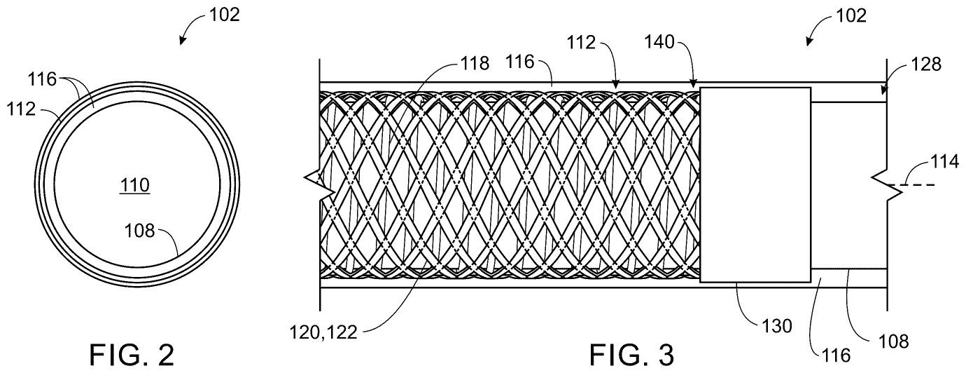

Referring to FIGS. 2 and 3, the tube 102 includes an inner conduit 108 (e.g., an inner tubular member) that forms a single lumen 110 of the catheter 100. The tube 102 also includes a reinforcement structure 112 that surrounds the inner conduit 108 along a central axis 114 of the tube 102 and an outer, external layer 116 (e.g., a coating) that surrounds both the inner conduit 108 and the reinforcement structure 112. Accordingly, the reinforcement structure 112 is positioned between (e.g., located intermediately of) the inner conduit 108 and the outer layer 116.

In some embodiments, the inner conduit 102 has an inner diameter of about 1.65 mm to about 1.70 mm (e.g., about 1.67 mm) and a wall thickness of about 0.027 mm to about 0.038 mm (e.g., about 0.033 mm). An inner surface of the inner conduit 108 is relatively smooth such that the inner conduit 108 enables substantially damage-free movement of the medical device through the lumen 110. The length of the tube 102 is that of the length of the inner conduit 108 and is generally selected to enable navigation throughout selected blood vessels to the target location within the patient's body. That is, the length may be selected to accommodate the patient's anatomical variations. The length may additionally be selected based on operational preferences of a physician utilizing the catheter 100. For example, an operational preference may relate to the compatibility of the tube 102 with other devices (e.g., a guide catheter or a guide wire), a preferred access location (e.g., radial access versus femoral access), or another parameter. In some embodiments, the length of the inner conduit 108 is within a range of about 85 cm to about 135 cm, depending on the target location within the body. For example, when the target location is within the neurovasculature, the length of the inner conduit 108 may be within a range of about 105 cm to about 125 cm, whereas when the target location is within the peripheral vasculature, the length of the inner conduit 108 may be within a range of about 85 cm to about 115 cm.

The material composition of the inner conduit 108 is such that the inner conduit 108 is relatively low in surface friction so as to minimize any resistance to inserting and navigating the medical device (e.g., a stent) within the inner conduit 108. This reduction in resistance during navigation minimizes the potential for trauma to vessel walls surrounding the tube 102. In some embodiments, the inner conduit 108 is made of polytetrafluoroethylene (PTFE). In some examples, PTFE is selected as the material for the inner conduit 108 due to its biocompatibility, chemical inertness, durability, and strength performance.

Additionally, in some embodiments, a hardness (e.g., and accordingly, a stiffness) of the inner conduit 108 varies along an axial length of the inner conduit 108. For example, the inner conduit 108 may include multiple sections (e.g., eight sections or another number of sections), each made of PTFE, that respectively have different, discreet measures of hardness (e.g., durometer). In some embodiments, each successive section of the inner conduit 108 (e.g., moving from the proximal end 106 of the tube 102 to distal end 128 of the tube 102) decreases in durometer (e.g., increases in softness).

The reinforcement structure 112 is a dual-layer structure that provides the tube 102 with resistance to radial deformation as the medical device is moved axially through the inner conduit 108 and exerts outwardly directed radial forces against an inner surface of the inner conduit 108. The reinforcement structure 112 includes two components that together provide the tube 102 with such structural integrity. For example, the reinforcement structure 112 includes a coiled wire 118 that surrounds the inner conduit 108 and a braided wire assembly 120 that surrounds the coiled wire 118. Together, the coiled wire 118 and braided wire assembly 120 advantageously improve the performance of the methodology for delivering the medical device by minimizing stretching and compression of the inner conduit 108 while the medical device is advanced through the inner conduit 108. In some embodiments, the reinforcement structure 112 is spaced apart from a distal end 128 of the tube 102 by a distance of about 0.5 mm to about 1.5 mm (e.g., about 1.0 mm) and otherwise extends proximally along the remaining length of the inner conduit 108.

Referring still to FIGS. 2 and 3, the coiled wire 118 is positioned directly on (e.g., in contact with) the inner conduit 108 such that the coiled wire 118 has an overall cylindrical shape. For example, during manufacture of the tube 102, the coiled wire 118 may be applied directly onto the inner conduit 108 via a coil winding machine, which results in a relatively high strength of the coiled wire 118 as compared to an otherwise similar coiled wire that would be formed separately and then placed over the inner conduit 108. The coiled wire 118 is a solid (e.g., non-hollow) component and is a key factor in imparting radial strength to the tube 102. While a medical device is advanced through the tube 102, the coiled wire 118 serves to maintain a substantially circular cross-sectional shape of the inner conduit 108.

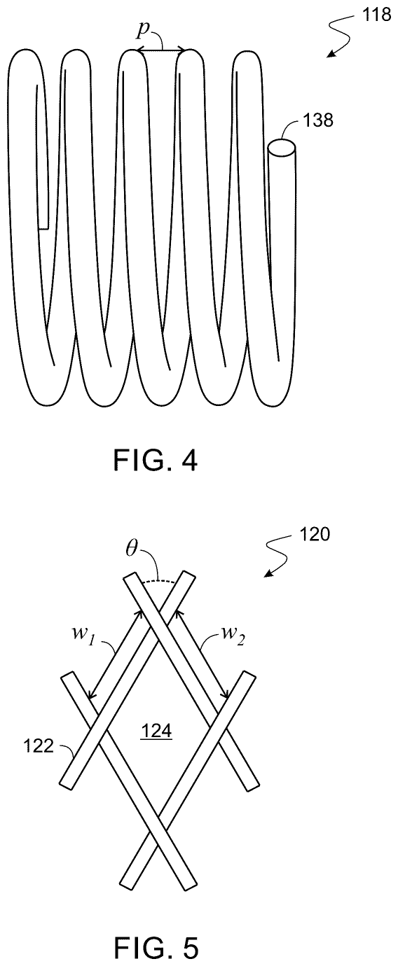

Referring to FIG. 4, in some embodiments, the coiled wire 118 has a pitch p (e.g., a gap between adjacent circumferential portions of the coiled wire 118) of about 0.15 mm to about 0.20 mm (e.g., about 0.18 mm). A core of the coiled wire 118 may have a circular cross-sectional shape 138 or the coiled wire 118 may have a non-circular (e.g., rectangular or other) cross-sectional shape along its core. The coiled wire 118 has a core width (e.g., a diameter or other width) of about 0.025 mm to about 0.088 mm (e.g., about 0.063 mm). The coiled wire 118 is made of one or more materials, such as stainless steel or nitinol. In some examples, the coiled wire 118 may be formed using a manufacturing process that utilizes a tension or winding angle during a coiling process to control the strength and/or stiffness of the coiled wire 118.

Referring to FIGS. 3 and 5, the braided wire assembly 120 is an interlocked, meshed structure. Accordingly, the braided wire assembly 120 includes multiple wires 122 that overlap each other in a regular pattern. The braided wire assembly 120 has an overall cylindrical shaped and is positioned directly on (e.g., in contact with) the coiled wire 118. For example, during manufacture of the tube 102, the braided wire assembly 120 may be applied directly onto the coiled wire 118 via a braiding machine, which results in a relatively strong internal tension of the braided wire assembly 120 as compared to an otherwise similar braided wire assembly that would be formed on a separate mandrel and then placed over the coiled wire 118. Such internal tension aids the coiled wire 118 in resisting radial deformation.

In some embodiments, spacings between the wires 122 of the braided wire assembly 120 form multiple openings 124 (e.g., four-sided openings). The openings 124 have a width w₁ defined by adjacent portions of the wires 122 along a first direction and a width w₂defined by adjacent portions of the wires 122 along a second direction. In some embodiments, the first and second directions together define an acute angle θ. The widths w₁ and w₂ of the openings 124 may be different or may be substantially equal, as shown in FIGS. 3 and 5. In some examples, a pattern of the braided wire assembly 120 is defined according to a number of wires 122 (e.g., carriers) and according to a braid density, or a number of pics per inch (PPI). In some examples, the PPI is defined as the number of times that the wires 122 cross over each other in the direction along each one inch of length. In some embodiments, the braided wire assembly 120 includes 16 wires and has a braid density of 65 PPI to 75 PPI (e.g., 70 PPI). In some embodiments, the number of wires 122 and the PPI determine the widths w₁ and w₂ of the openings 124 and the angle θ.

A size and a shape of the openings 124 can affect the ease with which the tube 102 is able to bend without affecting an ability of the inner conduit 108 to resist ovalization or to otherwise resist collapse of its circularity. Relatedly, as the number of wires 122 (e.g., carriers) increases, a stiffness, a torque response, and a kink resistance (e.g., a resistance to ovalization or resistance to collapse of circularity) of the inner conduit 108 increases. In some examples, the torque response refers to the ability of the inner conduit 108 to transmit a rotational force applied to the proximal end 106 (e.g., near an operator), to the distal end 128 (e.g., disposed inside of the patient's body). For example, a good torque response means that rotational movements of the distal end 128 closely mirror respective rotational movements of the proximal end 106 with minimal lag or loss of rotational accuracy. Additionally, as the PPI increases, the stiffness, the torque response, and a kink resistance of the inner conduit 108 increases.

Still referring to FIGS. 3 and 5, the wires 122 of the braided wire assembly 120 are solid (e.g., non-hollow), elongate, flat components that have a substantially rectangular cross-sectional shape. In some embodiments, each wire 122 has a width of about 2.54 μm to about 127 μm (e.g., about 76.2 μm) and a thickness of about 12.7 μm to about 50.8 μm (e.g., about 25.4 μm). The wires 122 are made of one or more materials, such as stainless steel or another material.

Referring to FIGS. 1 and 3, the tube 102 also includes multiple marker bands 130 that are spaced at strategic axial positions along the central axis 114 of the tube 102. The marker bands 130 are visible during medical imaging (e.g., digital subtraction angiography or any other suitable medical imaging technique) to provide visualization of the tube 102 as the tube 102 is moved through a blood vessel. The marker bands 130 can also aid in accurate visual estimation of the distal and proximal ends of the medical device relative to the target location. Accordingly, the marker bands 130 improves the physician's ability to estimate a landing position of the medical device when the device is fully deployed at the target location.

In some embodiments, a marker band 130 is located at (e.g., overlaps) a distal end 140 of the reinforcement structure 112. An additional one or more marker bands 130 may be located proximal to the distal-most marker band 130 and accordingly overlap the reinforcement structure 112. In some embodiments, the tube 102 includes a total of three marker bands 130. In other embodiments, the tube 102 may include a different number of marker bands 130. In some embodiments, each marker band 130 has a length (e.g., extending along the central axis 114 of the tube 102) of about 1 mm to about 3 mm (e.g., about 1.5 mm) and a thickness of about 0.025 mm to about 0.064 mm (e.g., about 0.038 mm). In some embodiments, the marker bands 130 are spaced apart from each other by a distance of about 2 cm to about 6 cm (e.g., about 4 cm). The marker bands 130 may be made of one or more radiopaque materials that enable visualization, such as one or more of platinum, iridium, and gold.

Referring to FIGS. 1 and 3, the outer layer 116 surrounds the above-discussed components of the tube 102 and thus forms the surface of the tube 102 that contacts the body. For example, the outer layer 116 may be formed as a hydrophilic coating that is fused over the reinforcement structure 112, the marker bands 130, and an outer surface of the inner conduit 108 to encapsulate and seal these components to form the tube 102.

The outer layer 116 is formed from one or more relatively soft polymer materials that provide only a minimum resistance to bending. Example materials from which the outer layer 116 may be made include polyurethane, thermoplastic elastomers (e.g., polyamide and polyether), and nylon materials. In some embodiments, the outer layer 116 is translucent or substantially transparent. In some embodiments, the outer layer 116 has a thickness within a range of about 0.33 mm to about 0.46 mm (e.g., about 0.38 mm). The thickness of the outer layer 116 may be at a minimum value along the reinforcement structure 112 and/or the marker bands 130 and at a maximum value along a portion of the tube 102 where the reinforcement structure 112 is not present. In this manner, the outer layer 116 imparts a substantially constant outer diameter to the tube 102.

As shown in FIGS. 2 and 3, the inner conduit 108, reinforcement structure 112, marker bands 130, and outer layer 116 are concentrically arranged and are maintained in position by the outer layer 116. The inner diameter of the tube 102 is that of the inner conduit 108, and the outer diameter of the tube 102 is defined by the concentric arrangement of the inner conduit 108, reinforcement structure 112, marker bands 130, and outer layer 116. In some embodiments, the outer diameter of the tube 102 is within a range of about 1.80 mm to about 1.90 mm (e.g., about 1.85 mm), such that the catheter 100 is a 5 French catheter. In some embodiments, the outer diameter of the tube 102 greatest along the marker bands 130 and is otherwise substantially constant along the remaining length of the tube 102. The combined attributes of the inner conduit 108, reinforcement structure 112, and outer layer 116 provide the catheter tube 102 with a construction that has a very low stiffness in flexion while maintaining a strong resistance to radial deformation (e.g., while maintaining a capability to minimize or substantially prevent radial deformation).

Referring again to FIG. 1, a catheter system 150 includes the catheter 100 and a navigation device 160 that is designed to cooperate with the catheter 100 to help guide the catheter 100 to the target location within the body. The navigation device 160 includes a main shaft portion 162, a distal shaft portion 164, and a distal tip 166 that together define a single lumen 168 of the navigation device 160. The lumen 168 extends from a proximal end of the navigation device 160 to an opening 174 at a distal end 170 of the navigation device 160. The main shaft portion 162 and the distal shaft portion 164 have a substantially cylindrical shape, while the distal tip 166 has a substantially conical shape that tapers from a maximum diameter (e.g., that of the shaft portions 162, 164) to a minimum diameter at the distal end 170. The navigation device 160 is sized to be inserted into the catheter tube 102 (e.g., through the entry port 134 of the access hub 104) to an extent that the full distal tip 166 is exposed from the distal end 128 of the tube 102.

In some embodiments, the main and distal shaft portions 162, 164 have an inner diameter of about 0.48 mm to about 0.56 mm (e.g., about 0.51 mm) and an outer diameter of about 1.22 mm to about 1.32 mm (e.g., about 1.27 mm). In some embodiments, the minimum diameter of the distal tip 166 is about 0.71 mm to about 0.81 mm (e.g., about 0.76 mm). The shaft portions 162, 164 have a combined length that are approximately equal to the length of the tube 102. In some embodiments, the distal tip 166 has a length of about 19 mm to about 20 mm (e.g., about 20 mm).

The main shaft portion 162 of the navigation device 160 is made of one or more materials, such as polyurethane, thermoplastic elastomers (e.g., polyamide and polyether), and nylon. The distal shaft portion 164 and the distal tip 166 together form a distal section 172 and are made of one or more ultra-soft materials that facilitate movement of the navigation device 160 around acute bends in the vasculature without resistance or kinking. For example, the distal section 172 may be made of one or more materials, such as polyurethane.

Furthermore, in some embodiments, the distal section 172 may be made of polyurethane that is mixed with one or more radiopaque materials (e.g., barium sulfate (BaSO₄) that allow the distal section 172 to be visualized during imaging and while the navigation device 160 is navigated through the vasculature. Alternatively or additionally to the utilization of BaSO₄, the distal section 172 may be equipped with radiopaque marker bands (e.g., similar to the marker bands 130 and made of platinum and iridium) for visualization. The distal end 170 of the navigation device 160 is a blunt end that allows for movement of the navigation device 160 through the vasculature without damaging (e.g., puncturing, tearing, or otherwise damaging) the vasculature.

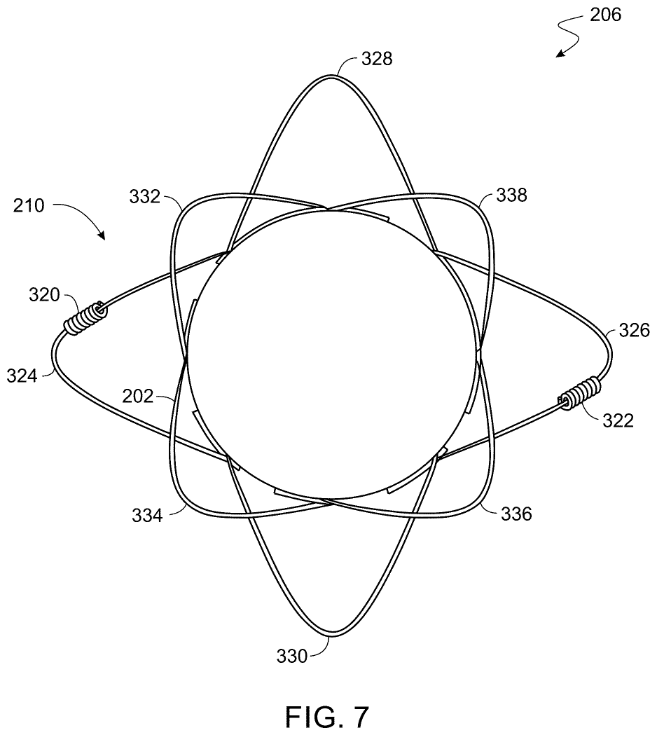

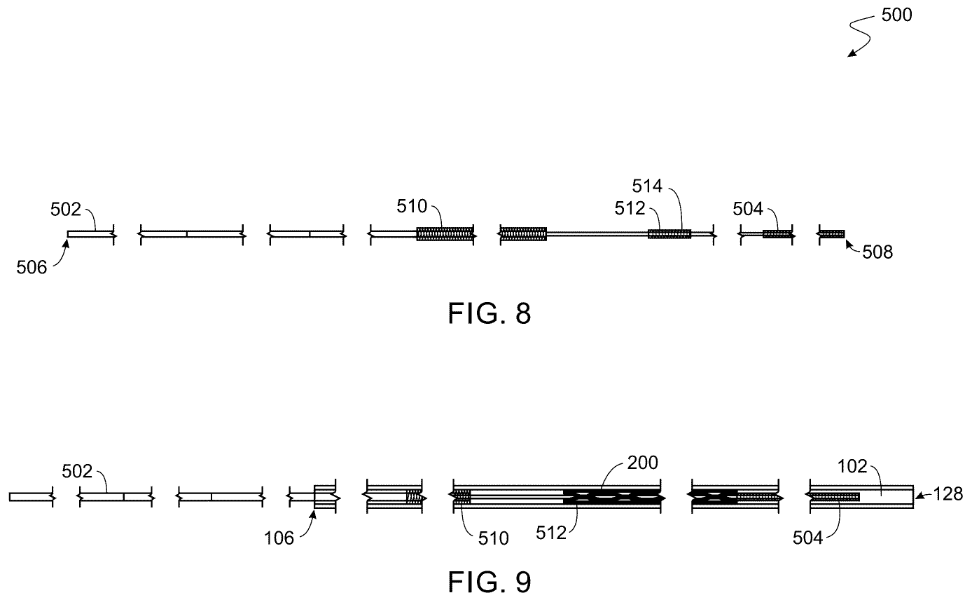

FIGS. 6 and 7 illustrate an example medical device that can be delivered to the target location using the catheter 100 such that the catheter 100, navigation device 160, insertion device 500 (discussed below with respect to FIGS. 8-10), and medical device together make up a medical intervention system. In this example, the medical device is embodied as a stent 200 that is designed to maintain a passageway through a portion of a blood vessel that is part of the neurovasculature so as to treat pulsatile tinnitus. The stent 200 includes a single wire 202 that is wound to form a braided body 204. The braided body 204 extends between a proximal end 206 of the stent 200 and a distal end 208 of the stent 200. In some embodiments, the wire 202 is a drawn filled tube (DFT) wire.

In some embodiments, a length of the stent 200 prior to implantation (e.g., an unconstrained length) is within a range about 20 mm to about 100 mm. The unconstrained length of the stent 200 may be selected based on the distance from the torcula of a patient through the transverse sinus and the sigmoid sinus of the patient such that the stent 200 can be deployed within these vessels while minimizing the risk of stenosis development following placement of the stent 200. In some embodiments, a diameter of the stent body 204 prior to implantation (e.g., an unconstrained diameter) is within a range of about 6 mm to about 10 mm and may be selected based on the anatomy of the patient. For example, the unconstrained diameter of the stent body 204 may be determined based on the diameter of the blood vessels of the patient's dural venous sinuses.

The wire 202 can be radiopaque in order to enable visualization of the stent 200 using any of the above-mentioned medical imaging techniques. Intersections of the wire 202 in the braided stent body 204 are not connected to one another, which enables crisscrossing portions of the wire 202 to slide past one another when the stent 200 moves along curves or is subjected to other forms of compression. Due to this structure of movable picks along the braided stent body 204, the stent 200 can remain patent (e.g., open and unobstructed) and resist kinking when positioned along curving pathways. The stent 200 is also heat treated such that the stent 200 has shape memory. Accordingly, the stent 200 can be compressed (e.g., inside of the catheter tube 102) during delivery of the stent 200 to the target blood vessel and can self-expand into the desired shape to contact the blood vessel walls once implanted.

While implanted, the stent 200 applies a chronic (e.g., continuous) force that is directed in a radially outward direction to the wall of the blood vessel. Such force maintains the blood vessel in a state that is sufficiently open to change the blood flow through the blood vessel from a pulsatile flow or turbulent flow to a laminar flow. As a result, the chronic force applied by the stent 200 following implantation in a blood vessel of the cerebral dural venous sinuses is sufficient to eliminate or significantly reduce the pulsatile tinnitus experienced by a patient. In some embodiments, the chronic force applied by the stent 200 following implantation is within a range of about 0.020 N/mm to about 0.050 N/mm.

Still referring to FIGS. 6 and 7, the stent 200 includes a flared crown 210, 212 at each of the proximal end 206 and the distal end 208 of the stent 200. The flared crowns 210, 212 allow the stent 200 to be secured to the surrounding blood vessel. Accordingly, the stent 200 can be deployed and maintained in position within a vein, such as within the cerebral dural venous sinuses of a patient. The unconstrained diameter of each of the crowns 210, 212 is typically within a range of about 1 mm to about 2 mm larger than the unconstrained diameter of the stent body 204. For example, in some embodiments, the unconstrained diameter of each of the crowns 210, 212 is in a range of about 7 mm to about 12 mm.

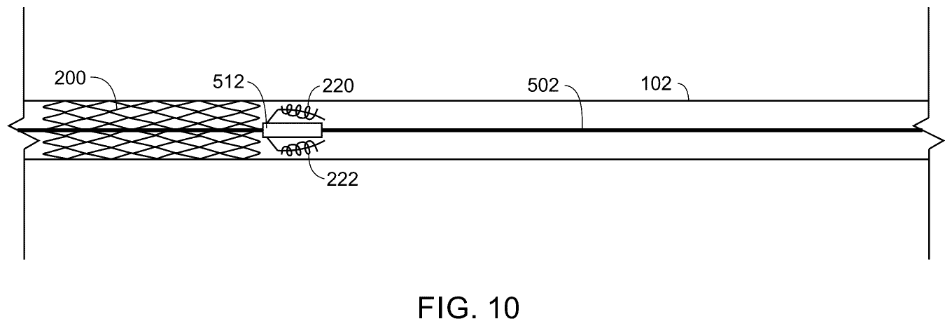

Referring to FIGS. 8 and 9, the stent 200 is configured to be implanted in the cerebral dural venous sinuses of a patient using an insertion device 500 and the catheter 100. The insertion device 500 includes a pusher rod 502, a stent body coil 504 positioned at a distal end 508 of the pusher rod 502, a radiopaque pusher body coil 510 positioned over a portion of the pusher rod 502, and a polymer tube 512 positioned over the pusher rod 502 between the stent body coil 504 and the radiopaque pusher body coil 510. In some embodiments, the pusher rod 502 tapers between the proximal end 506 of the pusher rod 502 and the distal end 508 of the pusher rod 502.

The stent body coil 504 of the insertion device 500 is attached to the proximal end the of pusher rod 502. The stent body coil 504 is formed of a coil of metal wire and is sufficiently flexible to prevent injury to the blood vessel of the patient as the insertion device 500 is advance through the patient's vasculature during implantation of the stent 200. The stent body coil 504 is configured to extend through about half of the length of the stent 200 when the stent 200 is loaded onto the insertion device 500 for implantation. The stent body coil 504 provides column strength and kink resistance to the pusher rod 502 and the stent 200 as the pusher rod 502 and the stent 200 are advanced through the catheter tube 102 during delivery of the stent 200 to a target vessel.

The pusher body coil 510 is attached to the pusher rod 502 between the proximal end 506 of the pusher rod 502 and the polymer tube 512. The pusher body coil 510 is formed of a coil of metal wire that is sufficiently flexible to prevent kinking of the pusher rod 502 as the pusher rod 502 is advanced through the patient's vasculature during implantation of the stent 200. In some embodiments, the pusher body coil is formed of stainless steel (e.g., 304V stainless steel), nitinol (NiTi), a platinum-tungsten alloy, platinum, or a combination thereof. In some embodiments, the pusher body coil 510 is formed of a radiopaque material, such as platinum. As a result, the pusher body coil 510 can be used during an implantation procedure in order to track the position of the insertion device 500 within the patient's vasculature throughout the implantation procedure using medical imaging. As a result, the clinician can monitor the position of the pusher body coil 510 within the patient's blood vessel in real time to ensure accurate placement of the stent 200.

Referring to FIG. 9, the polymer tube 512 is attached to the pusher rod 502 along the length of the pusher rod 502 between the stent body coil 504 and the pusher body coil 510. In some embodiments, a platinum tungsten (PtW) coil 514 is coupled to the pusher rod 502 underneath the polymer tube 512 such that the position of the polymer tube 512 can be tracked throughout an implantation procedure using medical imaging. The polymer tube 512, together with the catheter tube 200, are configured to releasably couple the proximal end 206 of the stent 200 to the pusher rod 502 during implantation of the stent 200. Therefore, by tracking the position of the polymer tube 512 during the implantation procedure, the position of the proximal end 206 of the stent 200 can be determined in real-time throughout the implantation procedure.

Referring to FIGS. 6-9, the stent 200 includes two marking coils 320, 322 positioned on the proximal end 206 of the stent body 204 and configured to interact with the polymer tube 512 and the catheter tube 102 to maintain the position of the stent 200 along the pusher rod 502 prior to implantation of the stent 200 in a blood vessel. The marking coils 320, 322 are each formed of a length of wire that is coiled around opposite loops 324, 326 of the stent body 304 at the proximal end 206 of the stent body 204. For example, the proximal end 206 of the stent body 204 includes four long loops 324, 326, 328, 330 and four short loops 332, 334, 336, 338, and the marking coils 320, 322 are coupled to the stent body 204 along two of long loops 324, 326 opposite each other. In some embodiments, the marking coils 320, 322 are soldered to the stent body 204. In some embodiments, the marking coils 320, 322 are glued to the stent body 204 using UV-curable adhesives or epoxy. In some embodiments, the marking coils 320, 322 are formed of radiopaque wire, which allows the marking coils 320, 322 to be visualized using medical imaging in real-time during implantation of the stent 200. Therefore, the position of the proximal end 206 of the stent 200 can be determined by tracking the position of the marking coils 320, 322 using medical imaging during the implantation procedure. In some embodiments, the marking coils 320, 322 are formed of DFT wire. For example, the marking coils 320, 322 can be formed of a nickel-titanium alloy (e.g., nitinol) tube that is filled with platinum, a platinum-tungsten alloy, gold, barium sulfate, or a combination thereof.

Referring to FIG. 10, when the stent 200 is loaded onto the insertion device 500 for implantation, the marking coils 320, 322 are positioned over and frictionally engage with the polymer tube 512. During implantation of the stent 200, the insertion device 500, with the stent 200 releasably coupled thereto, is inserted into and passed through the catheter tube 102. As the insertion device 500 and stent 200 are passed through the catheter tube 102, the marking coils 320, 322 are in contact with and positioned between the polymer tube 512 and the tube 102 (e.g., an inner surface of the inner conduit 108). The marking coils 320, 322 extend proximally beyond the polymer tube 512 and engage with a proximal surface of the polymer tube 512. The friction generated by the contact between the marking coils 320, 322 and the polymer tube 512 and the catheter tube 102 prevents movement of the stent 200 within the catheter tube 102 while the tube 102 is positioned over the marking coils 320, 322 and polymer tube 512. Once the distal end 128 of the catheter tube 102 is properly positioned within a blood vessel for delivery of the stent 200, the catheter tube 102 is withdrawn proximally while the position of the pusher rod 502 is maintained. Once the catheter tube 102 is withdrawn proximally over the polymer tube 512 such that the polymer tube 512 and the marking coils 320, 322 are positioned outside the catheter tube 102, the proximal end 206 of the stent body 204 expands, which disengages the marking coils 320, 322 from the polymer tube 512. As a result, the stent 200 is released from the insertion device 500 and is implanted within the blood vessel (e.g., the target location).

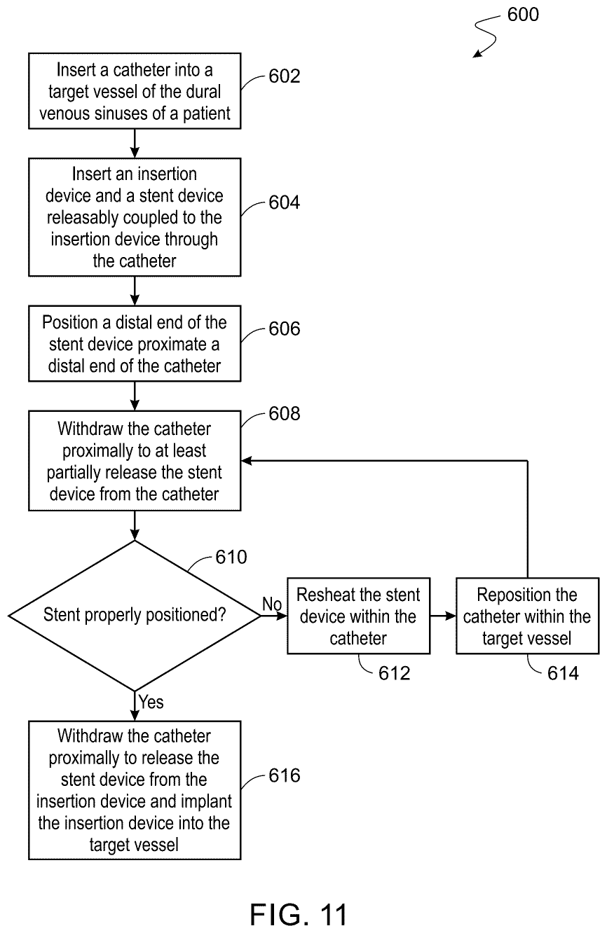

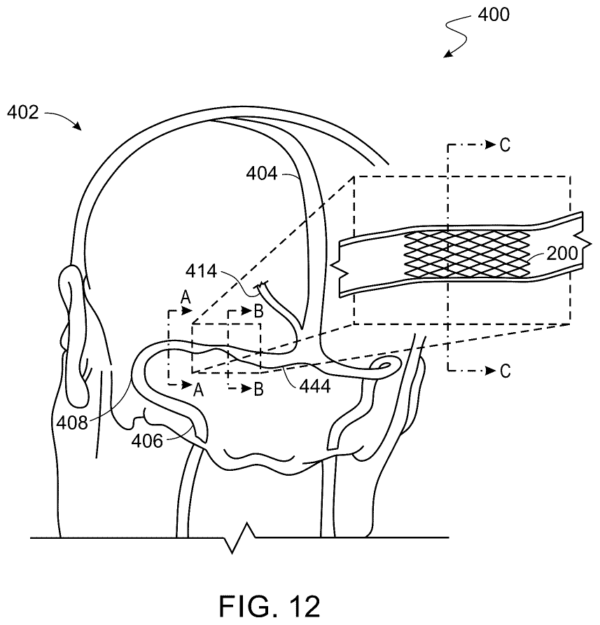

FIG. 11 is a flow chart illustrating an example method 600 of implanting a stent (e.g., the stent 200) in a patient using a catheter (e.g., the catheter 100). Referring to FIGS. 11 and 12, in some embodiments, the method 600 includes a step 602 of inserting a catheter tube (e.g., the catheter tube 102 in a target vessel of the cerebral dural venous sinuses 400 of a patient. The portions of the transverse sinus 444 that are not experiencing stenosis have a lumen that is generally wider than portions of the transverse sinus 444 that are experiencing stenosis, which have a reduced-width lumen. In order to implant the stent 200 within the transverse sinus 444, the catheter tube 102 is passed through the jugular bulb 406 and the sigmoid sinus 408 and into the transverse sinus.

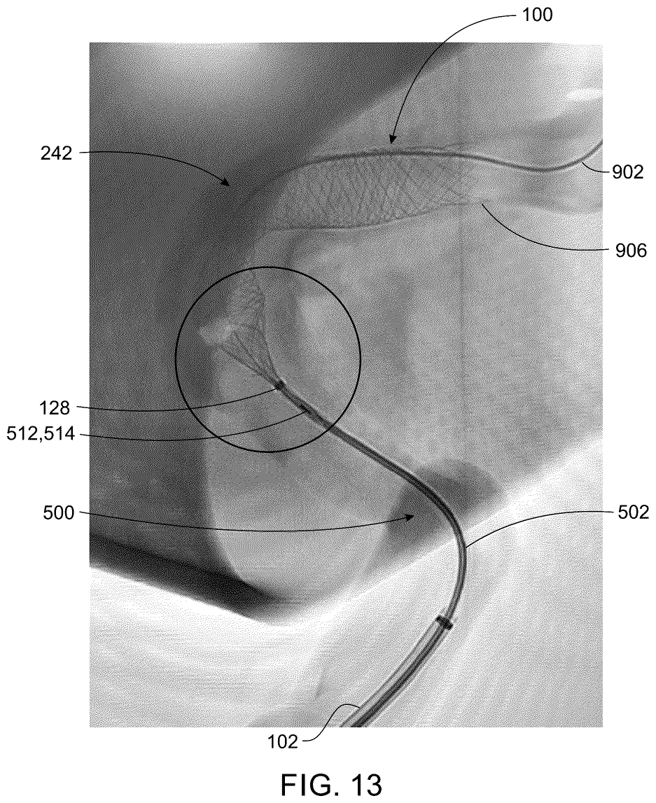

Referring to FIG. 13, in some embodiments, inserting the catheter tube 102 into a target vessel 906 is performed by accessing the femoral vein of the patient 402 using a micro-puncture kit, placing a short sheath in the femoral vein of the patient 402, and guiding the catheter tube 102 through the short sheath into the femoral vein using both the catheter 100 and the navigation device 160 (not illustrated in FIG. 13) and guidewire 902. As discussed above with respect to FIG. 1, using the navigation device 160 involves inserting the device 160 through the access hub 102 and into the catheter tube 102 until the distal tip 166 of the navigation device 160 extends from the distal end 128 of the tube 102. Once inserted into the femoral vein, the guidewire 902, navigation device 160, and tube 102 can be tracked using medical imaging (e.g., fluoroscopy) and advanced through the body to the target vessel 906 (e.g., the transverse sinus 444).

In some implementations, the catheter tube 102 is inserted into the target blood vessel 906 without the insertion device 500 and/or without the stent 200. For example, the catheter tube 102 may be placed in the target blood vessel 906 prior to inserting the insertion device 500 or the stent 200 into the tube 102.

Once the catheter tube 102 is inserted into the target vessel, the navigation device 160 is removed (e.g., withdrawn) from the catheter tube 102. An insertion device (e.g., the insertion device 500) and the stent, releasably coupled to the insertion device, are then inserted into the catheter (604), as shown in FIG. 13.

Referring again to FIG. 8, the stent 200 is coupled to the insertion device 500 by inserting the pusher rod 502 through the lumen of the stent 200 until the marking coils 320, 322 at the proximal end 206 of the stent 200 are in contact with and positioned at least partially over the polymer tube 512. Once the stent 200 is coupled to the pusher rod 502 with the marking coils 320, 322 positioned over the polymer tube 512, the pusher rod 502 is inserted into the proximal end 106 of the catheter tube 102.

Once inserted into the catheter tube, the insertion device together with the stent, is advanced distally through the catheter tube until the distal end of the stent is positioned proximate the distal end of the catheter tube (606). As discussed above with respect to FIG. 3, the dual-layer configuration of the reinforcement structure 112 of the catheter tube 102 causes the tube 102 to resist radial deformation as the stent 200 is moved axially through the tube 102 and exerts outwardly directed radial forces against an inner surface of the tube 102. As the pusher rod 502 is advanced distally through the catheter tube 102, the friction generated by the contact of the marking coils 320, 322 with the inner surface of the catheter tube 102 and with the polymer tube 512 is sufficient to prevent the stent 200 from moving within the catheter tube 102 relative to the pusher rod 502 while the stent 200 is advanced through the catheter tube 102. As a result, the stent 200 can be advanced and retracted within the catheter tube 102 via movement of the pusher rod 502 so long as the marking coils 320, 322 remain in contact with the inner surface of the catheter tube 102 and with the polymer tube 512.

Once the distal end of the catheter is properly positioned within the target vessel and the distal end of the stent is positioned proximate the distal end of the catheter tube, the catheter tube is withdrawn proximally to at least partially release the stent from the catheter (608). FIG. 13 depicts an example of real-time medical imaging of the stent 200 during implantation of the stent 200. As illustrated, as the catheter tube 102 is withdrawn proximally, the portion 242 of the stent 200 that is no longer contained inside of the catheter tube 102 self-expands to apply a force to the target vessel 906. As previously discussed, the stent 200 is formed of radiopaque DFT wire 102 and, as a result, the position of the expanded stent 200 can be monitored in real-time during the implantation procedure.

Additionally, the PtW coil 514 underneath the polymer tube 512 is radiopaque, and since the proximal end 206 of the stent 200 is coupled to the polymer tube 512 during insertion via marking coils 320, 322, the position of the proximal end 206 of the stent 200 can be tracked during implantation by monitoring the position of the polymer tube 512. Furthermore, in some embodiments, the marking coils 320, 322 are formed of a radiopaque material, such as radiopaque DFT wire, and the proximal end 206 of the stent 200 can be tracked during implantation by monitoring the position of the marking coils 320, 322 using medical imaging. By monitoring the position of the polymer tube 512 and/or the proximal end 206 of the stent 200 within the catheter tube 102, a clinician can determine how close the distal end 128 of the catheter tube 102 is from the proximal end 206 of the stent 200. This relates to how close the stent 200 is from being fully deployed from the catheter tube 102 and, as a result, no longer resheathable into the catheter tube 102.

Prior to fully deploying the stent from the catheter tube, a clinician uses medical imaging to visualize the position of the stent within the target vessel to determine whether the stent is properly positioned within the target vessel (610). If the clinician determines that the stent 200 is not properly positioned within the target vessel 906, the clinician can advance the catheter tube 102 distally to resheath the stent 200 within the catheter tube 102 (612), and the catheter tube 102 can be repositioned within the target vessel 906 (614). Once the catheter tube 102 is repositioned within the target vessel 906, the process of partially releasing the stent 200 from the catheter tube 102 and checking the positioning of the stent 200 can be repeated.

In addition, due to the self-expanding material used to form the stent 200 (e.g., nitinol), a clinician can use the catheter tube 102 to apply forward (e.g., distally directed) pressure on the portion of the stent 200 that has been deployed from the catheter tube 102, without resheathing the stent 200. By applying forward pressure on the deployed portion of the stent 200, the stent 200 can be positioned to more closely appose the walls of the target blood vessel 906 and better contour to the target blood vessel 906. In addition, by applying forward pressure on the stent 200 using the catheter tube 102 while the stent 200 is partially deployed, the stent 200 can be used to further open a stenosis of the target blood vessel 905 during deployment of the stent 200 within the blood vessel 906.

As previously discussed, the friction generated by the contact between the marking coils 320, 322 and the inner surface of the catheter tube 102 and the polymer tube 512 is sufficient to prevent the stent 200 from moving relative to the pusher rod 502 while being advanced through the catheter tube 102. As a result, the stent 200 can be resheathed into the catheter tube 102 so long as the marking coils 320, 322 and the polymer tube 512 are contained within the catheter tube 102. In some implementations, the stent 200 can be resheathed within the catheter tube 102 with up to 90% of the length of the stent body 204 deployed from the catheter tube 102. Thus, the stent 200 can be resheathed within the catheter tube 102 when 90% or less of the length of the stent body 204 has been deployed from the catheter tube 102. The ability to resheath the stent 200 into the catheter tube 102 provides several advantages, including enabling the clinician to reposition the stent 200 when the initial deployment location was improperly selected, when the stent 200 slips distally or proximally during deployment, or if the diameter or length of the selected stent 200 is determined to be improper based on the patient's anatomy.

Once the clinician has confirmed that the stent is properly positioned within the target vessel, the clinician withdraws the catheter tube proximally to release the stent from the insertion device and implant the stent within the target vessel (616). As previously discussed, the friction generated by the contact of the marking coils 320, 322 with the inner surface of the catheter tube 102 and the polymer tube 512 is sufficient to prevent the stent 200 from moving relative to the pusher rod 502. In order to release the stent 200 from the insertion device 500, the catheter tube 102 is withdrawn proximally while maintaining the position of the pusher rod 502 until the marking coils 320, 322 and polymer tube 512 are deployed out of the distal end 128 of the catheter tube 102 and positioned outside of the catheter tube 102. Once the proximal end 206 of the stent 200 is outside of the catheter tube 102, the proximal end 206 expands such that the marking coils 320, 322 are no longer frictionally engaged with the polymer tube 512. Thus, deploying the marking coils 320, 322 and polymer tube 512 outside the catheter tube 102 releases the stent 200 from the insertion device 500 and implants the stent 200 inside the target vessel 906. In some implementations, the force required to deploy the stent 200 outside the catheter tube 102 is in a range of about 1 N to about 5 N.

Once the stent 200 is deployed from the catheter tube 102, the stent 200 self-expands to an expanded state within the target vessel 906 in the patient's dural venous sinuses. The stent 200 contacts the inner walls of the target vessel 906 and applies force to the blood vessel 906 that is sufficient to change the blood flow within the blood vessel 906 from pulsatile or turbulent flow to laminar flow. For example, the stent 200 contacts and applies a chronic outward force to the inner wall of the transverse sinus to cause the lumen of the transverse sinus to increase to allow for increased blood flow through the transverse sinus 444. In some embodiments, following implantation of the stent 200, the lumen of the transverse sinus 444 is expanded to achieve a height of about 8 mm and a width of about 6 mm. As a result, the chronic outward force provided by the stent 200 following implantation in the target vessel 906 of the cerebral dural venous sinuses is sufficient to eliminate or significantly reduce the pulsatile tinnitus experienced by a patient. Once the stent 200 is fully deployed from the catheter tube 102 and expanded within the lumen of the target blood vessel 906 (i.e., once the stent 200 is implanted in the blood vessel), the contact between the stent 200 and the inner walls of the blood vessel, and in particular the contact between the flared crowns 310, 312 of the stent 200 and the blood vessel, permanently fixes the position of the stent 200 within the blood vessel. Therefore, the stent 200 cannot be resheathed or removed from the blood vessel 906 once the stent 200 has been fully deployed from the catheter tube 102 and implanted within the blood vessel.

Once the stent 200 is released from the insertion device 500, the insertion device 500 can be withdrawn proximally through the catheter tube 102 and removed from the patient. Once the stent 200 is implanted in the target vessel 906 and the insertion device 500 has been removed, the catheter tube 102 can be further withdrawn proximally and removed from the patient.

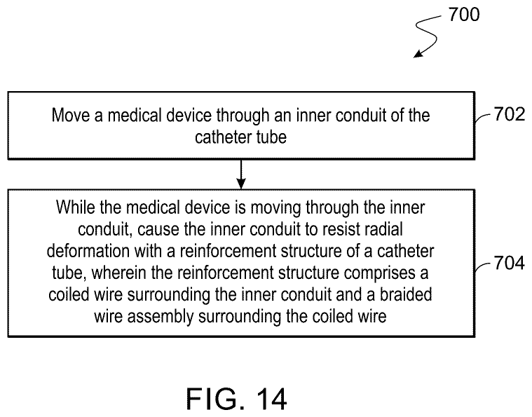

FIG. 14 is a flow chart illustrating an example method 700 of performing a medical intervention. In some embodiments, the method 700 includes a step 702 for moving a medical device (e.g., the stent 200) through an inner conduit (e.g., the inner conduit 108) of a catheter tube (e.g., the tube 102). In some embodiments, the method 700 includes a step 704 for causing the inner conduit to resist radial deformation with a reinforcement structure (e.g., the reinforcement structure 112) of the catheter tube while the medical device is moving through the inner conduit, wherein the reinforcement structure includes a coiled wire (e.g., the coiled wire 118) surrounding the inner conduit and a braided wire assembly (e.g., the braided wire assembly 120) surrounding the coiled wire.

While the catheter 100, navigation device 160, and stent 200 have been described and illustrated with respect to certain dimensions, sizes, shapes, arrangements, materials, and methods 600, in some embodiments, a catheter, navigation device, or stent that is otherwise respectively similar in construction and/or function to the catheter 100, navigation device 160, or stent 200 may include one or more different dimensions, sizes, shapes, arrangements, configurations, or materials, or be operated according to different methods. For example, while the catheter 100 has been described with respect to the method 600 of delivering the stent 200 to a neurovascular target location and with respect to the method 700 of performing a medical intervention, in some implementations, the catheter 100 may be used as a guide or support catheter for other interventional neuroradiology procedures.

Accordingly, other embodiments are within the scope of the following claims.

1. A catheter tube comprising:
an inner conduit having a smooth inner surface to provide for a substantially damage-free movement of a medical device through a lumen of the inner conduit;
a reinforcement structure comprising:
a coiled wire having a circular core cross-sectional shape, having a pitch of about 0.15 mm to about 0.20 mm, and surrounding the inner conduit, and
a braided wire assembly surrounding the coiled wire,
wherein the reinforcement structure is configured to cause the inner conduit to resist radial deformation as a medical device is moved through the inner conduit, and
wherein a distal end of the reinforcement structure is spaced apart from a distal end of the inner conduit;
a marker band configured to provide visualization of the catheter tube while the catheter tube is disposed within a patient's body, the marker band surrounding an outer surface of the reinforcement structure at the distal end of the reinforcement structure such that the marker band is spaced apart from the distal end of the inner conduit; and
an outer layer surrounding the marker band, the reinforcement structure, and the inner conduit in a concentric arrangement.

2. The catheter tube of claim 1, wherein the catheter tube is a 5 French catheter tube.

3. The catheter tube of claim 1, wherein a hardness of the inner conduit varies along a length of the inner conduit.

4. (canceled)

5. The catheter tube of claim 1, wherein the braided wire assembly comprises a plurality of wires that overlap each other in a regular pattern to form a plurality of openings.

6. The catheter tube of claim 5, wherein the plurality of wires comprises 16 wires.

7. The catheter tube of claim 6, wherein the braided wire assembly has a braid density of 65 pics per inch (PPI) to 75 PPI.

8. The catheter tube of claim 1, further comprising one or more additional marker bands.

9. The catheter tube of claim 8, wherein the marker band and the one or more additional marker bands comprise a total of three marker bands that are equally spaced from one another.

10. The catheter tube of claim 8, wherein the one or more additional marker bands surround the outer surface of the reinforcement structure.

11. The catheter tube of claim 8, wherein each of the marker band and the one or more additional maker bands comprises one or more radiopaque materials.

12. The catheter tube of claim 1, wherein the outer layer is fused to the reinforcement structure and to an outer surface of the inner conduit to encapsulate the reinforcement structure and the outer surface of the inner conduit.

13. The catheter tube of claim 12, wherein the outer layer comprises a coating, and wherein the coating forms an outer-most surface of the catheter tube such that the coating is configured to contact an adjacent structure within a patient's body.

14. The catheter tube of claim 1, wherein the inner conduit defines the lumen, and wherein the lumen comprises a single lumen that is sized to accommodate the medical device.

15. A medical intervention system comprising:
an implantable medical device; and
a catheter tube comprising:
an inner conduit having a smooth inner surface to provide for a substantially damage-free movement of the implantable medical device through a lumen of the inner conduit,
a reinforcement structure comprising:
a coiled wire having a circular core cross-sectional shape, having a pitch of about 0.15 mm to about 0.20 mm, and surrounding the inner conduit, and
a braided wire assembly surrounding the coiled wire,
wherein the reinforcement structure is configured to cause the inner conduit to resist radial deformation as the implantable medical device is moved through the inner conduit, and
wherein a distal end of the reinforcement structure is spaced apart from a distal end of the inner conduit, and
a marker band configured to provide visualization of the catheter tube while the catheter tube is disposed within a patient's body, the marker band surrounding an outer surface of the reinforcement structure at the distal end of the reinforcement structure such that the marker band is spaced apart from the distal end of the inner conduit, and
an outer layer surrounding the marker band, the reinforcement structure, and the inner conduit in a concentric arrangement.

16. The medical intervention system of claim 15, wherein the implantable medical device comprises a stent that is configured to be implanted at a target location within the patient's body.

17. The medical intervention system of claim 16, wherein the target location comprises a portion of a blood vessel within the patient's neurovasculature.

18. The medical intervention system of claim 15, further comprising a navigation device sized to be positioned within the catheter tube.

19. The medical intervention system of claim 18, wherein the navigation device comprises a main shaft and a radiopaque distal section.

20. The medical intervention system of claim 19, wherein the radiopaque distal section comprises a substantially cylindrical distal shaft and a tapered distal tip that is configured to extend from a distal end of the catheter tube while the catheter tube is moved through the patient's body.

21. A method of performing a medical intervention, the method comprising:
moving an implantable medical device through a lumen of an inner conduit of a catheter tube within a patient's body, wherein the inner conduit has a smooth inner surface to provide for a substantially damage-free movement of the implantable medical device through the lumen; and
while the implantable medical device is moving through the inner conduit, causing the inner conduit to resist radial deformation with a reinforcement structure of the catheter tube, wherein a distal end of the reinforcement structure is spaced apart from a distal end of the inner conduit, and wherein the reinforcement structure comprises:
a coiled wire having a circular core cross-sectional shape, having a pitch of about 0.15 mm to about 0.20 mm, and surrounding the inner conduit, and
a braided wire assembly surrounding the coiled wire; and
visualizing the catheter tube within the patient's body at a radiopaque marker band surrounding an outer surface of the reinforcement structure at the distal end of the reinforcement structure, wherein the radiopaque marker band is spaced apart from the distal end of the inner conduit, and
wherein the catheter tube further comprises an outer layer that surrounds the radiopaque marker band, the reinforcement structure, and the inner conduit in a concentric arrangement.

22. The method of claim 21, further comprising moving the implantable medical device past a distal end of the catheter tube to implant the medical device at a target location within a patient's body.

23. The method of claim 22, wherein the target location comprises a portion of a blood vessel of the neurovasculature.

24. The method of claim 21, wherein the implantable medical device comprises a stent.

25. The method of claim 21, wherein the catheter tube is a 5 French catheter tube.

26. The method of claim 21, further comprising causing the inner conduit to maintain a substantially circular cross-sectional shape while the implantable medical device is moved through the inner conduit.

27. (canceled)

28. The method of claim 27, wherein the catheter tube further comprises one or more additional radiopaque marker bands.

29. The method of claim 21, further comprising, prior to moving the implantable medical device through the inner conduit of the catheter tube:
placing a navigation device within the catheter tube; and
guiding the catheter tube to a target location within the patient's body using the navigation device.

30. The method of claim 29, wherein the navigation device comprises a main shaft and a radiopaque distal section, and wherein the radiopaque distal section comprises a substantially cylindrical distal shaft and a tapered distal tip that is configured to extend from a distal end of the catheter tube while the catheter tube is in use.

31. The catheter tube of claim 5, wherein each wire of the plurality of wires comprises a flat wire.

32. (canceled)

33. The medical intervention system of claim 15, wherein the implantable medical device comprises a braided stent, and wherein the smooth inner surface of the inner conduit is configured to facilitate substantially damage-free movement of the braided stent through the lumen of the inner conduit.

34. The medical intervention system of claim 15, wherein the implantable medical device is configured to be released from the distal end of the inner conduit to be implanted at a target location within the patient's body.

35. The catheter tube of claim 1, wherein the distal end of the reinforcement structure is spaced apart from the distal end of the inner conduit by a distance of about 0.5 mm to about 1.5 mm.
